(19) United States
(12) Patent Application Publication
(10) Pub. No.: US 2022/0135522 A1
(43) Pub. Date: May 5, 2022

(54) A PROCESS FOR OBTAINING 4,4'-DICHLORODIPHENYL SULFOXIDE

(71) Applicant: BASF SE, Ludwigshafen am Rhein (DE)

(72) Inventors: Lukas METZGER, Ludwigshafen am Rhein (DE); Christian SCHUETZ, Ludwigshafen am Rhein (DE); Indre THIEL, Ludwigshafen am Rhein (DE); Stefan BLEI, Ludwigshafen am Rhein (DE)

(21) Appl. No.: 17/429,028

(22) PCT Filed: Feb. 6, 2020
(86) PCT No.: PCT/EP2020/052968

§ 371 Date: Aug. 6, 2021

(30) Foreign Application Priority Data
Feb. 8, 2019 (EP) 19156178.6

(51) Int. Cl. C07C 315/06

(52) U.S. Cl. CPC C07C 315/06

(57) ABSTRACT

The invention relates to a process for obtaining 4,4′-dichlorodiphenyl sulfoxide from a liquid mixture comprising dichlorodiphenyl sulfoxide and a solvent, comprising: (a) cooling the liquid mixture to a temperature below the saturation point of 4,4′-dichlorodiphenyl sulfoxide in the solvent to obtain a suspension comprising crystallized 4,4′-dichlorodiphenyl sulfoxide, (b) solid-liquid-separation of the suspension to obtain a residual moisture containing solid 4,4′-dichlorodiphenyl sulfoxide as a product and mother liquor, (c) concentrating the mother liquor, (d) recycling at least a part of the concentrated mother liquor into the cooling step (a).

The invention relates to a process for obtaining 4,4′-dichlorodiphenyl sulfoxide from a liquid mixture comprising 4,4′-dichlorodiphenyl sulfoxide and a solvent. 4,4′-dichlorodiphenyl sulfoxide (in the following also termed DCDPSO) also is called 1-chloro-4(4-chlorophenyl)sulfinyl benzene or bis(4-chlorophenyl)sulfoxide.

DCDPSO can be used as a precursor for producing 4,4-dichlorodiphenyl sulfone which is used for example as a monomer for preparing polymers such as polyarylene ethers like polysulfone, polyether sulfone, or polyphenylene sulfone or as an intermediate of pharmaceuticals, dyes and pesticides.

A liquid mixture comprising DCDPSO and a solvent generally emanates from a production process of DCDPSO. It is further possible to produce the liquid mixture by mixing DCDPSO and solvent, for example for purification of DCDPSO.

For the production of DCDPSO several processes are known. One process is a Friedel-Crafts reaction with thionyl chloride and chlorobenzene as starting materials in the presence of a catalyst, for example aluminum chloride. Generally the reaction of thionyl chloride and chlorobenzene is disclosed as a first part in the production of 4,4′-dichlorodiphenyl sulfone, whereby an intermediate reaction product is obtained by the reaction of thionyl chloride and chlorobenzene which is hydrolyzed at an elevated temperature and thereafter oxidized to yield 4,4′-dichlorodiphenyl sulfone.

General processes for the production of sulfur containing diary compounds are disclosed for example in Sun, X. et al, “Investigations on the Lewis-acids-catalysed electrophilic aromatic substitution reactions of thionyl chloride and selenyl chloride, the substituent effect, and the reaction mechanisms”, Journal of Chemical Research 2013, pages 736 to 744, Sun, X. et al, “Formation of diphenyl sulfoxide and diphenyl sulfide via the aluminum chloride-facilitated electrophilic aromatic substitution of benzene with thionyl chloride, and a novel reduction of sulfur(IV) to sulfur(II)”, Phosphorus, Sulfur, and Silicon, 2010, Vol. 185, pages 2535-2542 and Sun, X. et al., “Iron(II) chloride (FeCl₃)-catalyzed electrophilic aromatic substitution of chlorobenzene with thionyl chloride (SOCl₂) and the accompanying auto-redox in sulfur to give diaryl sulfides (Ar₂S): Comparison to catalysis by aluminum chloride (AlCl₃)”, Phosphorus, Sulfur, and Silicon, 2017, Vol. 192, No. 3, pages 376 to 380. In these papers different reaction conditions and catalysts are compared.

Friedel-Crafts acylation reactions of thionyl chloride and chlorobenzene in the presence of Lewis acid catalyst as part in the production of 4,4′-dichlorodiphenylsulfone are also disclosed for instance in CN-A 108047101, CN-A 102351756, CN-A 102351757, CN-A 102351758 or CN-A 104557626.

A two-stage process for producing 4,4′-dichlorodiphenyl sulfone, where in the first stage DCDPSO is produced, is disclosed in CN-B 104402780. For producing DCDPSO, a Friedel-Crafts reaction is described to be carried out at 20 to 30° C. using thionyl chloride and chlorobenzene as raw material and anhydrous aluminum chloride as catalyst. The Friedel-Crafts reaction is followed by cooling, hydrolysis, heating and refluxing. It is further described that after reflux is finished the reaction mixture is cooled down and DCDPSO precipitates in form of white crystals which are filtered off. The DCDPSO then is oxidized to obtain 4,4′-dichlorodiphenyl sulfone.

SU-A 765262 also discloses a two-stage process for producing 4,4′-dichlorodiphenyl sulfone where in the first stage DCDPSO is obtained by a Friedel-Crafts reaction using thionyl chloride and chlorobenzene in the presence of aluminum chloride at a temperature in the range from -10 to 50° C. According to the examples, the mixture obtained in the Friedel-Crafts reaction is poured into a 3% aqueous solution of hydrochloric acid and heated to completely dissolve the DCDPSO in the chlorobenzene which is added in excess. After separation into two phases, the organic phase is washed and then cooled to precipitate the DCDPSO. In one example the hydrochloric acid is obtained by trapping the hydrogen chloride evolved in the Friedel-Crafts reaction.

It is an object of the present invention to provide a process for obtaining 4,4′-dichlorodiphenyl sulfoxide from a liquid mixture comprising DCDPSO and a solvent, which allows an efficient separation of the DCDPSO from the solvent with a good yield, environmental sustainability and which is energy efficient.

This object is achieved by a process for obtaining DCDPSO from a liquid mixture comprising DCDPSO and a solvent (in the following termed as “liquid mixture”), comprising:
(a) cooling the liquid mixture to a temperature below the saturation point of DCDPSO in the solvent to obtain a suspension comprising crystallized DCDPSO,
(b) solid-liquid-separation of the suspension to obtain a residual moisture containing solid DCDPSO as a product and mother liquor,
(c) concentrating the mother liquor,
(d) recycling at least a part of the concentrated mother liquor into the cooling step (a).

By cooling the majority of the DCDPSO crystallizes, however, still a remarkable part of the DCDPSO remains solved in the solvent. By concentrating the mother liquor and recycling the mother liquor into the cooling step (a) it is possible to obtain most of the DCDPSO solved in the solvent and thus reduce the amount of product removed from the process.

The saturation point denotes the temperature of the liquid mixture at which DCDPSO starts to crystallize. This temperature depends on the concentration of the DCDPSO in the liquid mixture. The lower the concentration of DCDPSO in the liquid mixture, the lower is the temperature at which crystallization starts.

The solvent used in the liquid mixture can be any solvent in which DCDSPO is sufficiently soluble, in particular at a temperature suitable for industrial scale production, and from which crystallized DCDPSO can be separated in a convenient manner. Such solvent is for example chlorobenzene, toluene, xylene, mesitylene, methanol or a mixture of two or more of said solvents. As DCDPSO generally emanate from the manufacture of DCDPSO, the solvent used in the liquid mixture preferably is chlorobenzene, particularly monochlorobenzene.

Cooling (a) for crystallizing DCDPSO can be carried out in any crystallization apparatus or any other apparatus which allows cooling of the liquid mixture, for example an apparatus with surfaces that can be cooled such as a vessel or a tank with cooling jacket, cooling coils or cooled baffles like so called “power baffles”.

Cooling of the liquid mixture for crystallization of the DCDPSO can be performed either continuously or batchwise. To avoid precipitation and fouling on cooled surfaces, it is preferred to carry out the cooling in a gastight closed vessel by
(i) reducing the pressure in the gastight closed vessel;
(ii) evaporating solvent;
(iii) condensing the evaporated solvent by cooling;
(iv) returning the condensed solvent into the gastight closed vessel.

This process allows for cooling the liquid mixture without cooled surfaces onto which crystallized DCDPSO accumulates and forms a solid layer. This enhances the efficiency of the cooling process. Also, additional efforts to remove this solid layer can be avoided. Therefore, it is particularly preferred to use a gastight closed vessel without cooled surfaces.

To avoid precipitation of the crystallized DCDPSO it is further preferred to agitate the liquid mixture in the crystallization apparatus. Therefore, a suitable apparatus is for example a stirred tank or draft-tube crystallizer. If the crystallization apparatus is a stirred tank, any stirrer can be used. The specific power input into the crystallizer by the stirring device preferably is in the range from 0.2 to 0.5 W/kg, more preferred in the range from 0.2 to 0.35 W/kg. Preferably, a stirrer type is used which leads to a rather homogeneous power input without high gradients concerning local energy dissipation.

To crystallize the DCDPSO, it is preferred to provide crystal nuclei. To provide the crystal nuclei, it is possible to use dried crystals which are added to the liquid mixture or to add a suspension comprising particulate DCDPSO as crystal nuclei. If dried crystals are used but the crystals are too big, it is possible to grind the crystals into smaller particles which can be used as crystal nuclei. Further, it is also possible to provide the necessary crystal nuclei by applying ultrasound to the liquid mixture. Preferably, the crystal nuclei are generated in situ in an initializing step. The initializing step preferably comprises the following steps before setting the reduced pressure in step (i):
reducing the pressure in the gastight closed vessel such that the boiling point of the liquid mixture is in the range from 80 to 95° C.;
evaporating solvent until an initial formation of solids takes place;
increasing the pressure in the vessel and heating the liquid mixture in the vessel to a temperature in the range from 85 to 100° C.

By reducing the pressure in the vessel such that the boiling point of the liquid mixture is in the range from 80 to 95° C., more preferred in the range from 83 to 92° C., the following evaporation of solvent leads to a saturated solution and the precipitation of DCDPSO. By the following pressure increase and heating the liquid mixture in the gastight closed vessel to a temperature in the range from 85 to 100° C., the solidified DCDPSO starts to partially dissolve again. This has the effect that the number of crystal nuclei is reduced, which allows producing a smaller amount of crystals with a bigger size. Cooling, particularly by reducing the pressure, can be started immediately after a pre-set temperature within the above ranges is reached to avoid complete dissolving of the produced crystal nuclei. However, it is also possible to start cooling after a dwell time, for example of 0.5 to 1.5 h at the pre-set temperature.

For generating the crystal nuclei in the initializing step, it is possible to only evaporate solvent until an initial formation of solids take place. It is also possible to entirely condense the evaporated solvent by cooling and to return all the condensed solvent into the gastight closed vessel. The latter has the effect that the liquid in the gastight closed vessel is cooled and solid forms. A mixture of both approaches, where only a part of the evaporated and condensed solvent is returned into the gas tight vessel, is also viable.

If the cooling and thus the crystallization of DCDPSO is performed batchwise, it is preferred to carry out steps (ii) to (iv) during the pressure reduction in step (i). Thereby, it is particularly preferred to continuously reduce the pressure in step (i) until the temperature in the gastight closed vessel reaches a predefined value in the range from 0 to 45° C., preferably in the range from 10 to 35° C. and particularly in the range from 20 to 30° C. At these predefined temperatures the pressure in the gastight closed vessel typically is in the range from 20 to 350 mbar(abs), more preferred in the range from 20 to 200 mbar(abs) and particularly in the range from 20 to 100 mbar(abs). After the predefined temperature value is reached, pressure reduction is stopped and then the gastight closed vessel is vented until ambient pressure is reached. The temperature profile in the gastight closed vessel preferably is selected such that the liquid mixture is subjected to a constant supersaturation. These conditions can be achieved by adapting the cooling profile while keeping the temperature below the saturation temperature at the respective concentration of DCDPSO in the liquid phase. In detail the adapted cooling profile is chosen based on phase equilibria, mass of crystal nuclei, and initial size of the crystal nuclei. Further, to adapt the cooling profile, constant grow rates are assumed. To determine the data for adapting the cooling profile, for example turbidity probes, refractive index probes or ATR-FTIR-probes can be used. The temperature profile and/or pressure profile for example can be stepwise, linear or progressive.

To reduce the solubility of the DCDPSO and thus increase the yield of solidified DCDPSO it is necessary to shift the saturation point. This is possible by continuously reducing the amount of solvent at a constant temperature, for example by evaporating solvent, or by cooling the liquid mixture at constant concentration. Since reduction of the amount of solvent results in a very viscous suspension when a certain critical concentration is reached, it is preferred to increase the yield of solidified DCDPSO partly by reducing the amount of solvent by evaporation followed by reducing the temperature. For reducing the solubility of DCDPSO in the liquid mixture and to improve the crystallization, it is possible to additionally add at least one drowning-out agent, for example at least one protic solvent like water, an alcohol, and/or an acid, particularly a carboxylic acid, or at least one highly unpolar solvent like a linear and/or cyclic alkane. With respect to ease of workup water, methanol, ethanol, acetic acid and/or formic acid, particularly water and/or methanol are preferred drowning-out agents.

After reaching ambient pressure the suspension comprising particulate 4,4′-dichlorodiphenyl sulfoxide in a solvent (in the following termed as “suspension”) which formed in the gastight closed vessel by the cooling is withdrawn and fed into the solid-liquid-separation (b).

If the cooling and thus the crystallization of DCDPSO is performed continuously, it is preferred to operate the cooling and crystallization stepwise in at least two steps, particularly in two to three steps. If the cooling and crystallization is carried out in two steps, in a first step the liquid mixture preferably is cooled to a temperature in the range from 40 to 90° C. and in a second step preferably to a temperature in the range from −10 to 50° C. If the cooling is operated in more than two steps, the first step preferably is operated at a temperature in the range from 40 to 90° C. and the last step at a temperature in the range from −10 to 30° C. The additional steps are operated at temperatures between these ranges with decreasing temperature from step to step. If the cooling and crystallization is performed in three steps, the second step for example is operated at a temperature in the range from 10 to 50° C.

As in the batchwise process, the temperature in the continuously operated process can be set by using apparatus for cooling and crystallization having surfaces to be cooled, for example a cooled jacket, cooling coils or cooled baffles like so called “power baffles”. To establish the at least two steps for cooling and crystallization, for each step at least one apparatus for cooling and crystallization is used. To avoid precipitation of DCDPSO, also in the continuous process it is preferred to reduce the temperature by reducing the pressure in the apparatus for cooling and crystallization wherein the apparatus for cooling and crystallization preferably are gastight closed vessels. Suitable apparatus for cooling and crystallization for example are agitated-tank crystallizers, draft-tube crystallizers, horizontal crystallizers, forced-circulation crystallizers or Oslo-crystallizers. The pressure which is set to achieve the required temperature corresponds to the vapor pressure of the liquid mixture. Due to the pressure reduction, low boilers, particularly solvent, evaporate. The evaporated low boilers are cooled to condense, and the condensed low boilers are returned into the respective apparatus for cooling and crystallization by which the temperature is set.

If the cooling and crystallization is carried out continuously, a stream of the suspension is continuously withdrawn from the apparatus for cooling and crystallization. The suspension then is fed into the solid-liquid-separation (b). To keep the liquid level in the apparatus for cooling and crystallization within predefined limits, fresh liquid mixture comprising DCDPSO and solvent can be fed into the apparatus in an amount corresponding or essentially corresponding to the amount of suspension withdrawn from the apparatus. The fresh liquid mixture either can be added continuously or batchwise each time a minimum liquid level in the apparatus for cooling and crystallization is reached.

Independently of being carried out batchwise or continuously, crystallization preferably is continued until the solids content in the suspension in the last step of the crystallization is in the range from 5 to 50 wt %, more preferred in the range from 5 to 40 wt % and particularly in the range from 20 to 40 wt %, based on the mass of the suspension.

Even though the cooling and crystallization can be carried out continuously or batchwise, it is preferred to carry out the cooling and crystallization batchwise and particularly to cool the liquid mixture by reducing the pressure according to the above described process comprising steps (i) to (iv) to avoid precipitation of crystallized DCDPSO on cooled surfaces of an apparatus for cooling and crystallization. Batchwise cooling and crystallization allows a higher flexibility in terms of operating window and crystallization conditions and is more robust against variations in process conditions.

Independently of whether the cooling and crystallization is performed continuously or batchwise, the solid-liquid-separation (b) can be carried out either continuously or batchwise, preferably continuously.

If the cooling and crystallization is carried out batchwise and the solid-liquid-separation is carried out continuously, at least one buffer container is used into which the suspension withdrawn from the apparatus used for cooling and crystallization is filled. For providing the suspension a continuous stream is withdrawn from the at least one buffer container and fed into a solid-liquid-separation apparatus. The volume of the at least one buffer container preferably is such that each buffer container is not totally emptied between two filling cycles in which the contents of the apparatus for cooling and crystallization are fed into the buffer container. If more than one buffer container is used, it is possible to fill one buffer container while the contents of another buffer container are withdrawn and fed into the solid-liquid-separation. In this case the at least two buffer containers are connected in parallel. The parallel connection of buffer containers further allows filling the suspension into a further buffer container after one buffer container is filled. An advantage of using at least two buffer containers is that the buffer containers may have a smaller volume than only one buffer container. This smaller volume allows a more efficient mixing of the suspension to avoid sedimentation of the crystallized DCDPSO. To keep the suspension stable and to avoid sedimentation of solid DCDPSO in the buffer container, it is possible to provide the buffer container with a device for agitating the suspension, for example a stirrer, and to agitate the suspension in the buffer container. Agitating preferably is operated such that the energy input by stirring is kept at a minimal level, which is high enough to suspend the crystals but prevents them from breakage. For this purpose, the energy input preferably is in the range from 0.2 to 0.5 W/kg, particularly in the range from 0.25 to 0.4 W/kg.

If the cooling and crystallization and the solid-liquid-separation are carried out batchwise, the contents of the vessel for cooling and crystallization directly can be fed into a solid-liquid-separation apparatus as long as the solid-liquid separation apparatus is large enough to take up the whole contents of the vessel for cooling and crystallization. In this case it is possible to omit the buffer container. It is also possible to omit the buffer container when cooling and crystallization and the solid-liquid-separation are carried out continuously. In this case also the suspension directly is fed into the solid-liquid-separation apparatus. If the solid-liquid separation apparatus is too small to take up the whole contents of the vessel for cooling and crystallization, also for batchwise operation at least one additional buffer container is necessary to allow to empty the crystallization apparatus and to start a new batch.

If the cooling and crystallization are carried out continuously and the solid-liquid-separation is carried out batchwise, the suspension withdrawn from the cooling and crystallization apparatus is fed into the buffer container and each batch for the solid-liquid-separation is withdrawn from the buffer container and fed into the solid-liquid-separation apparatus.

The solid-liquid-separation for example comprises a filtration, centrifugation or sedimentation. Preferably, the solid-liquid-separation is a filtration. In the solid-liquid-separation liquid mother liquor is removed from the solid DCDPSO and residual moisture containing DCDPSO (in the following also termed as “moist DCDPSO”) is obtained. If the solid-liquid-separation is a filtration, the moist DCDPSO is called “filter cake”.

Independently of whether it is carried out continuously or batchwise, the solid-liquid-separation preferably is performed at ambient temperature or temperatures below ambient temperature, preferably at ambient temperature. It is possible to feed the suspension into the solid-liquid-separation apparatus with elevated pressure for example by using a pump or by using an inert gas having a higher pressure, for example nitrogen. If the solid-liquid-separation is a filtration and the suspension is fed into the filtration apparatus with elevated pressure, the differential pressure necessary for the filtration process is realized by setting ambient pressure to the filtrate side in the filtration apparatus. If the suspension is fed into the filtration apparatus at ambient pressure, a reduced pressure is set to the filtrate side of the filtration apparatus to achieve the necessary differential pressure. Further, it is also possible to set a pressure above ambient pressure on the feed side of the filtration apparatus and a pressure below ambient pressure on the filtrate side or a pressure below ambient pressure on both sides of the filter in the filtration apparatus, wherein also in this case the pressure on the filtrate side must be lower than on the feed side. Further, it is also possible to operate the filtration by only using the static pressure of the liquid layer on the filter for the filtration process. Preferably, the pressure difference between feed side and filtrate side and thus the differential pressure in the filtration apparatus is in the range from 100 to 6000 mbar(abs), more preferred in the range from 300 to 2000 mbar(abs) and particularly in the range from 400 to 1500 mbar(abs), wherein the differential pressure also depends on the filters used in the solid-liquid-separation (b).

To carry out the solid-liquid-separation (b) any solid-liquid-separation apparatus known by the skilled person can be used. Suitable solid-liquid-separation apparatus are for example an agitated pressure nutsche, a rotary pressure filter, a drum filter, a belt filter or a centrifuge. The pore size of the filters used in the solid-liquid-separation apparatus preferably is in the range from 1 to 1000 μm, more preferred in the range from 10 to 500 μm and particularly in the range from 20 to 200 μm.

Particularly preferably, cooling and crystallization is carried out batchwise and the solid-liquid-separation is operated continuously.

According to the invention, the mother liquor withdrawn from the solid-liquid separation apparatus, preferably the filtration apparatus, and thus depleted in 4,4′-dichlorodiphenyl sulfoxide is concentrated in step (c). Concentration of the mother liquor preferably is performed by distillation or evaporation, preferably by evaporation.

The distillation or evaporation for concentrating the mother liquor can be carried out either at ambient pressure or at the reduced pressure, preferably at a pressure in the range from 20 to 800 mbar(abs), more preferred in a range from 50 to 500 mbar(abs), and particularly in a range from 100 to 350 mbar(abs).

During the evaporation process low boilers, particularly solvent, evaporate and are withdrawn. DCDPSO which is a high boiler remains in the liquid mother liquor and thus the concentration of DCDPSO increases. The amount to which the mother liquor is reduced in the evaporation depends on the amount of DCDPSO in the mother liquor and the desired concentration in the concentrated mother liquor. The minimum amount to which the mother liquor can be reduced should be larger than the amount of DCDPSO in the mother liquor. Further, the minimum amount of low boiler which is evaporated should be such that the concentration of DCDPSO in the concentrated mother liquor rises. Thus, depending on the concentration of DCDPSO in the mother liquor, the evaporation process preferably is continued until the amount of mother liquor is reduced to 4 to 80 wt %, more preferred to 4 to 40 wt % and particularly to 4 to 20 wt % of the amount of mother liquor fed into the evaporation apparatus. Suitable evaporation apparatus for example are vessels, preferably stirred vessels, rotary evaporators, thin film evaporators and falling film evaporators. Particularly preferred the evaporation apparatus is a falling film evaporator.

Besides an evaporation process it is also possible to carry out a distillation process for concentrating the mother liquor. In a distillation process the low boilers comprising solvent are removed as a top stream. The concentrated mother liquor usually is withdrawn from the distillation process as a bottom stream. The distillation process for example is carried out in a distillation column. Suitable distillation columns for example are plate columns or packed columns. If packed columns are used, either packed beds or structured packings can be used. A suitable pressure for operating such a distillation column is for instance in the range from 20 mbar(abs) to 800 mbar(abs), preferably 50 to 500 mbar(abs), in particular 100 to 350 mbar(abs). The bottom temperature and the head temperature of the distillation column depend on the pressure and the bottom temperature preferably is in a range from 40 to 110° C., more preferred in a range from 55° C. to 100° C. and particularly in a range from 55 to 80° C. and the head temperature preferably is in a range from 30 to 100° C., more preferred in a range from 45 to 90° C. and particularly in a range from 45 to 80° C.

Evaporation or distillation preferably is continued until the concentration of DCDPSO in the mother liquor is in the range from 6 to 60 wt %, more preferred in the range from 10 to 50 wt %, and particularly in the range from 15 to 40 wt %, based on the total amount of the concentrated mother liquor.

At least a part of the concentrated mother liquor is recycled into the cooling (a). To avoid an excessive accumulation of high boiling byproducts and contaminants, it is preferred to recycle a part of the concentrated mother liquor into the cooling (a) and to withdraw the rest of the concentrated mother liquor from the process. The amount of concentrated mother liquor recycled into the cooling (a) preferably is in the range from 10 to 95 wt %, more preferred in the range from 40 to 90 wt %, and particularly in the range from 65 to 90 wt %, each based on the total amount of concentrated mother liquor.

If the cooling (a) is carried out batchwise, the concentrated mother liquor obtained from one batch preferably is recycled into the following batch.

The recycled concentrated mother liquor preferably is mixed with fresh liquid mixture and fed into the cooling (a). The ratio of fresh liquid mixture to concentrated mother liquor preferably is in the range from 60:1 to 6:1, more preferred in the range from 15:1 to 7:1 and particularly in the range from 10:1 to 7:1.

Mixing of the recycled concentrated mother liquor and the fresh liquid mixture can be carried out before feeding into the apparatus in which the cooling and crystallization takes place such that a mixture of recycled concentrated mother liquor and fresh liquid mixture is fed into the apparatus. Alternatively, the recycled concentrated mother liquor and the fresh liquid mixture are fed separately into the apparatus in which the cooling and crystallization takes place and are mixed in this apparatus.

The product of the process is residual moisture containing solid 4,4′-dichlorodiphenyl sulfoxide (in the following termed as “moist DCDPSO”). If the solid-liquid-separation is a filtration, the product is deposited on the filter of the filtration apparatus. The DCDPSO can be used as such for instance as insecticide. It is more typically used as a precursor for the production of other compounds, for instance in the field of pharmaceuticals or polymers. Generally the DCDPSO is used for producing 4,4′-dichlorodiphenyl sulfone in a following oxidation step. Prior to further use, such as feeding the DCDPSO into the oxidation step, it is possible to further treat the DCDPSO, for example in a purification step. For purification of the DCDPSO it is for example possible to repeat the crystallization and filtration. For this purpose, after filtration the DCDPSO is mixed with fresh solvent and heated to a temperature at which the DCDPSO solves in the solvent to achieve the liquid mixture of DCDPSO and solvent. This liquid mixture then is cooled to again crystallize the DCDPSO. An advantage of solving the DCDPSO and repeating cooling and crystallization (a), filtration (b), concentrating the mother liquor (c) and recycling at least part of the mother liquor (d) is that impurities which might be comprised in the crystallized DCDPSO can be removed and thus a higher purity of the product can be achieved. Besides repeating the process, the purification step also may comprise a washing step with a suitable washing liquid. A suitable washing liquid for example is the solvent which also is used to produce the liquid mixture of DCDPSO and solvent.

The liquid mixture comprising DCDPSO can originate from any process for producing DCDPSO in which a liquid mixture comprising DCDPSO and solvent is produced.

The liquid mixture can be obtained for example in a process for producing DCDPSO comprising:
(A) reacting thionyl chloride, chlorobenzene and aluminum chloride in a molar ratio of thionyl chloride:chlorobenzene:aluminum chloride of 1:(6 to 9):(1 to 1.5) at a temperature in the range from 0 to below 20° C., forming an intermediate reaction product and hydrogen chloride;
(B) mixing aqueous hydrochloric acid and the intermediate reaction product at a temperature in the range from 70 to 110° C. to obtain a crude reaction product comprising DCDPSO,
(C) separating the crude reaction product into an organic phase comprising the DCDPSO and an aqueous phase,
(D) washing the organic phase with an extraction liquid.

To obtain DCDPSO, in the reaction (A) thionyl chloride, chlorobenzene and aluminum chloride are fed into a reactor in a molar ratio of thionyl chloride:chlorobenzene:aluminum chloride of 1:(6 to 9):(1 to 1.5), preferably in a molar ratio of thionyl chloride:chlorobenzene:aluminum chloride of 1:(6 to 8):(1 to 1.2) and particularly in a molar ratio of thionyl chloride:chlorobenzene:aluminum chloride of 1:(6 to 7):(1 to 1.1).

The reactor can be any reactor which allows mixing and reacting of the components fed into the reactor. A suitable reactor is for example a stirred tank reactor or jet loop reactor. If a stirred tank reactor is used, the stirrer preferably is an axially conveying stirrer, for example an oblique blade agitator. The reaction can be operated either continuously or batchwise. Preferably, the reaction is operated batchwise.

The thionyl chloride, chlorobenzene and aluminum chloride can be added simultaneously or successively. For reasons of ease of conduct of the reaction—in particular in case of batch reaction—preferably, aluminum chloride and chlorobenzene are fed firstly into the reactor and then the thionyl chloride is added to the aluminum chloride and chlorobenzene. In this case the aluminum chloride and chlorobenzene can be added simultaneously or one after the other. However, in each case it is preferred to mix the aluminum chloride and chlorobenzene before adding the thionyl chloride. Particularly preferably aluminum chloride and chlorobenzene are first fed into the reactor and the thionyl chloride is added to the aluminum chloride and chlorobenzene. During the reaction hydrogen chloride (HCl)—typically in gaseous form—is formed which is at least partially withdrawn from the reactor. The volumetric flow for adding the thionyl chloride typically depends on heat dissipation and flow rate of the gas withdrawn from the reactor.

The chlorobenzene which is added in excess into the reactor and, therefore, only partially converted during the chemical reaction, also serves as a solvent for the reaction products.

The thionyl chloride and the chlorobenzene react in the presence of the aluminum chloride whereby an intermediate reaction product and hydrogen chloride form. The intermediate reaction product comprises 4,4′-dichlorodiphenyl sulfoxide-AICl₃ adduct. The aluminum chloride generally can act as catalyst. The chemical reaction can be schematically represented by the following chemical reaction equation (1):

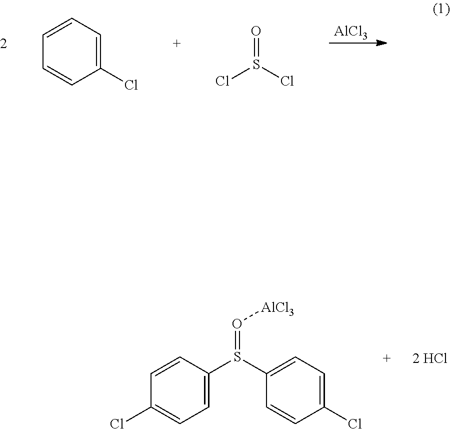

The reaction (A) is carried out at a temperature in the range from 0 to below 20° C., preferably at a temperature in the range from 3 to 15° C. and particularly in the range from 5 to 12° C.

Thereby the reaction can be carried out at a constant or almost constant temperature. It is also possible to carry out the reaction at varying temperatures within the described ranges, for instance employing a temperature profile over the time of reaction or the reactor.

The reaction period generally depends on the amount of reactants used and increases with increasing amounts of reactants. After addition of the thionyl chloride to the mixture of aluminum chloride and chlorobenzene is completed, the reaction preferably is continued for 10 to 120 min, more preferred from 20 to 50 min after the total amount of thionyl chloride is fed into the reactor. Independently of whether the reaction is operated continuously or batchwise, the flow rate of the thionyl chloride is selected such that the heat generated by the reaction can be dissipated from the reactor by suitable cooling devices to keep the temperature in the reactor within a predefined range.

The hydrogen chloride (HCl) produced in the reaction typically is in gaseous form and at least partly removed from the reactor. While it can be put to other use in gaseous form, preferably, the hydrogen chloride removed from the reaction is mixed with water to produce aqueous hydrochloric acid.

After the reaction the intermediate reaction product is mixed with aqueous hydrochloric acid. For reasons of energy as well as production efficiency as well as sustainability, particularly preferably, the aqueous hydrochloric acid is produced from the hydrogen chloride removed from the reaction (A). By mixing the intermediate reaction product with the aqueous hydrochloric acid, hydrolysis of the intermediate reaction product can take place. A crude reaction product comprising DCDPSO is obtained. The crude reaction product can also comprise aluminum chloride which is typically in hydrated form, usually as AlCl₃-6H₂O. The hydrolysis can be schematically represented by reaction equation (2):

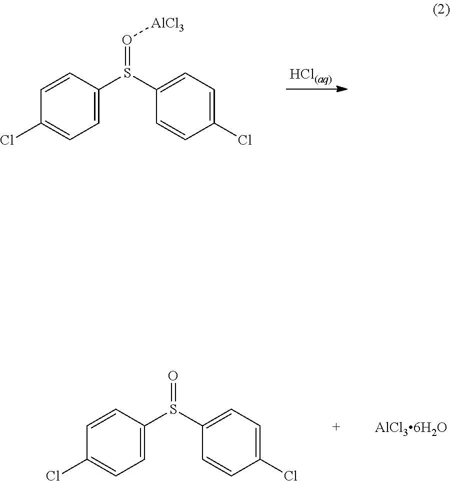

The temperature at which the hydrolysis is carried out is in the range from 70 to 110° C., preferably in the range from 80 to 100° C. and particularly in the range from 80 to 90° C. The reaction period of the hydrolysis after all components for the hydrolysis are added preferably is in the range from 30 to 120 min, more preferred in the range from 30 to 60 min and particularly in the range from 30 to 45 min. This reaction period is in general sufficient for hydrolysis of the intermediate reaction product to obtain the DCDPSO. To facilitate the hydrolysis and to bring it as fast as possible to completion, the mixture can be agitated, preferably the mixture is stirred. After finishing the hydrolysis the mixture separates into an aqueous phase comprising the AlCl₃ and an organic phase comprising DCDPSO solved in the excess chlorobenzene. In case the mixture is stirred, stirring is stopped to allow the mixture to separate.

The aqueous hydrochloric acid may have any concentration. However, a concentration of the hydrochloric acid above 3 wt % improves the solubility of the aluminum chloride. Preferably, the aqueous hydrochloric acid used in the hydrolysis has a concentration in the range from 3 to 12 wt %, more preferably in the range from 6 to 12 wt % and particularly preferably in the range from 10 to 12 wt %. All concentrations of hydrochloric acid in wt % above and in the following are based on the total amount of hydrogen chloride and water in the aqueous hydrochloric acid. An advantage of a higher concentration, particularly of a concentration in the range from 10 to 12 wt % is that the density of the aqueous phase increases and the aqueous phase thus forms the lower phase whereas the upper phase is the organic phase comprising the DCDPSO, in the following also termed as “organic phase”. This allows an easier draining of the aqueous phase to obtain the organic phase. Further, the higher concentration allows a smaller amount of water for removing the aluminum chloride. A higher concentration of the aqueous hydrochloric acid further results in a quicker phase separation.

The amount of aqueous hydrochloric acid used in (B) preferably is such that no aluminum chloride precipitates and that further two liquid phases are formed the lower phase being the aqueous phase and the organic phase being the upper phase. To achieve this, the amount of aqueous hydrochloric acid added to the reaction mixture preferably is such that after the hydrolysis the weight ratio of aqueous to organic phase is in the range from 0.6 to 1.5 kg/kg, more preferably in the range from 0.7 to 1.0 kg/kg and particularly in the range from 0.8 to 1.0 kg/kg. A smaller amount of aqueous hydrochloric acid may result in precipitation of aluminum chloride. Particularly at higher concentrations of the aqueous hydrochloric acid a larger amount is necessary to avoid precipitation. Therefore, the concentration of the aqueous hydrochloric acid preferably is kept below 12 wt %.

The reaction of thionyl chloride, chlorobenzene and aluminum chloride and the mixing with aqueous hydrochloric acid and thus the hydrolysis can be carried out in the same reactor or in different reactors. Preferably, the reaction is carried out in a first reactor and the hydrolysis in a second reactor. If a first reactor and a second reactor are used, the first reactor corresponds to the reactor as described above. The second reactor also can be any reactor to perform a batchwise reaction and which allows agitating, preferably stirring of the components in the reactor. Therefore, the second reactor also preferably is a stirred tank reactor.

Either the one reactor, if the reaction and the hydrolysis are carried out in the same reactor, or the preferably used first and second reactors is, respectively are designed in such a way that the temperature can be set to adjust the temperature in the reactor. For this purpose, it is for example possible to provide a pipe inside the reactor through which a heating medium or a cooling medium can flow. Under the aspect of ease of reactor maintenance and/or uniformity of heating, preferably, the reactor comprises a double jacket through which the heating medium or cooling medium can flow. Besides the pipe inside the reactor or the double jacket the heating and/or cooling of the reactor(s) can be performed in each manner known to a skilled person.

If the reaction and the hydrolysis are carried out in different reactors, it is particularly preferred to heat the intermediate reaction product to a temperature which is above the solubility point of the intermediate reaction product in the solvent after the reaction is completed and prior to transporting the intermediate reaction product from the first reactor to the second reactor. Due to heating the intermediate reaction product before transporting and feeding into the second reactor, the intermediate reaction product dissolves and a liquid without solid components is transported. This has the advantage that fouling of the first reactor is avoided.

The solubility point denotes the temperature of the reaction mixture at which the intermediate reaction product is fully dissolved in the solvent. This temperature depends on the concentration of the intermediate reaction product in the solvent. The lower the concentration of DCDPSO in the organic phase, the lower is the temperature at which the intermediate reaction product is fully dissolved in the solvent.

If the reaction and the hydrolysis are carried out in the same reactor, the aqueous hydrochloric acid is fed into the reactor after the reaction is completed and after the intermediate reaction product is heated to the temperature of the hydrolysis. The flow rate of the aqueous hydrochloric acid preferably is set such that the temperature of the hydrolysis can be held in the specified range for the hydrolysis by tempering the reactor. If the reaction and the hydrolysis are carried out in different reactors, it is preferred to firstly feed the aqueous hydrochloric acid into the sec- and reactor and to add the intermediate reaction product to the aqueous hydrochloric acid. In this case the flow rate of adding the intermediate reaction product into the second reactor is set such that the temperature in the second reactor is held within the specified temperature limits for the hydrolysis by tempering the second reactor.

To remove the aqueous hydrochloric acid and remainders of the aluminum chloride from the organic phase, the organic phase obtained in (C) is separated off and washed with an extraction liquid.

The phase separation following the hydrolysis can be carried out in the reactor in which the hydrolysis took place or in a separate vessel for phase separation. Under the aspect of less complexity, preferably the phase separation is carried out in the reactor in which the hydrolysis took place. After the phase separation is completed, the aqueous phase and the organic phase are removed separately from the vessel in which the phase separation took place, preferably the reactor in which the hydrolysis was performed. Using aqueous hydrochloric acid having a higher concentration for removing aluminum chloride, particularly aqueous hydrochloric acid having a concentration in the range from 10 to 12 wt % so that the density of the aqueous phase increases and the aqueous phase thus forms the lower phase, has the additional advantage that for the easier draining of the aqueous phase the washing of the organic phase can be carried out in the same apparatus as the hydrolysis.

After being separated off, the organic phase is fed into the washing step (D) to remove residual aluminum chloride and hydrochloric acid. The extraction liquid used for washing the organic phase preferably is water.

The washing preferably is carried out in a separate washing vessel. However, it is also possible to only remove the aqueous phase from the reactor in which the hydrolysis took place and carry out the washing step in the reactor in which the hydrolysis took place. If the washing is carried out in a separate washing vessel, any vessel in which an organic phase can be washed can be used. The washing vessel usually comprises means to intimately mix the organic phase with the extraction liquid. Preferably, the washing vessel is a stirred tank into which the organic phase and the extraction liquid are fed and then mixed.

If the phase separation is carried out in a vessel for phase separation, the washing either can be carried out in a washing vessel or, alternatively, in the vessel for phase separation. If phase separation and washing are carried out in the same vessel, it is necessary to provide means for mixing the organic phase with the extraction liquid after the aqueous phase which was separated from the organic phase is drained off.

The washing preferably is carried out at a temperature in the range from 70 to 110° C., more preferred in a range from 80 to 100° C. and particularly in a range from 80 to 90° C. Particularly preferably the washing is carried out at the same temperature as the hydrolysis.

Generally, the amount of extraction liquid which preferably is water is sufficient to remove all or essentially all of the aluminum chloride from the organic phase. Under the aspect of waste control it is usually preferred to use as little extraction liquid as possible. The amount of water used for washing preferably is chosen in such a way that a weight ratio of aqueous to organic phase in the range from 0.3 to 1.2 kg/kg, more preferably in the range from 0.4 to 0.9 kg/kg and particularly in the range from 0.5 to 0.8 kg/kg is obtained. In terms of sustainability and avoidance of large waste water streams it is preferred to use as little water for the washing step as possible. It is particularly preferred to use such an amount of water that the entire aqueous phase from the washing step can be used to generate the aqueous hydrochloric acid in the concentration needed for hydrolysis. For this purpose, the water which is used for washing is separated off and mixed with the hydrogen chloride obtained in the reaction to obtain the aqueous hydrochloric acid. The mixing of the hydrogen chloride and the water can be performed for example in a washing column into which the gaseous hydrogen chloride and the water are fed. If such a washing column is used, preferably the hydrogen chloride and the water are fed in countercurrent. Besides a washing column all further vessels which allow absorbing the hydrogen chloride in water can be used. Thus, it is possible for example to feed the water into a vessel and to introduce the hydrogen chloride into the water. To introduce the hydrogen chloride into the water, for example a pipe can be used which immerges into the water. For distributing the hydrogen chloride in the water, it is possible to provide the end of the pipe immerging into the water with an immersion head having small holes through which the hydrogen chloride flows into the water. As an alternative, also a frit can be used for distributing the hydrogen chloride in the water.

After a predetermined washing period, mixing is stopped to allow the mixture to separate into an aqueous phase and an organic phase. The aqueous phase and the organic phase are removed from the washing vessel separately. The organic phase comprises the liquid mixture comprising DCDPSO solved in the excess chlorobenzene as solvent. The predetermined washing period preferably is as short as possible to allow for short overall process times. At the same time, it needs sufficient time to allow for the removal of aluminum chloride.

The process may comprise one or more than one such washing cycles. Usually one washing cycle is sufficient.

Each process step described above can be carried out in only one apparatus or in more than one apparatus depending on the apparatus size and the amount of compounds to be added. If more than one apparatus is used for a process step, the apparatus can be operated simultaneously or—particularly in a batchwise operated process—at different time. This allows for example to carry out a process step in one apparatus while at the same time another apparatus for the same process step is maintained, for example cleaned. Further, in that process steps where the contents of the apparatus remain for a certain time after all components are added, for example the reaction or the hydrolysis, it is possible after feeding all compounds in one apparatus to feed the components into a further apparatus while the process in the first apparatus still continues. However, it is also possible to add the components into all apparatus simultaneously and to carry out the process steps in the apparatus also simultaneously.

An illustrative embodiment of the invention is shown in the figure and explained in more detail in the following description.

In the drawing:

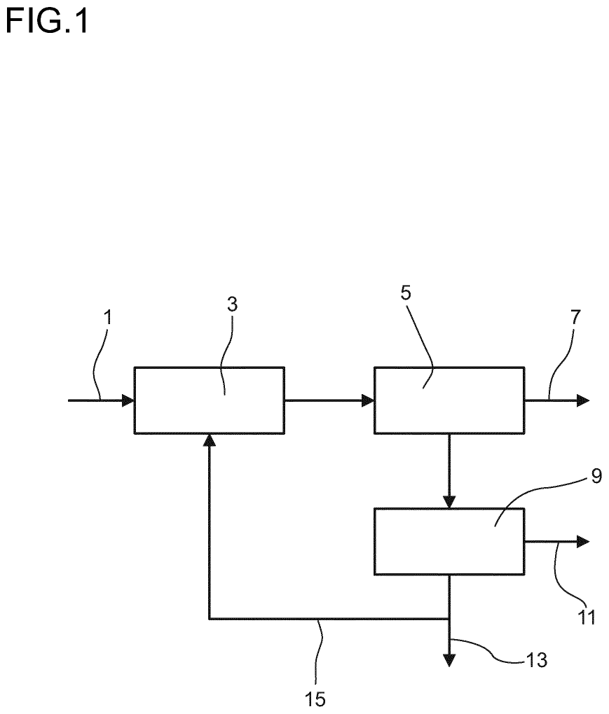

FIG. 1 shows a schematic flow diagram of the process for obtaining DCDPSO from a liquid mixture comprising DCDPSO and solvent,

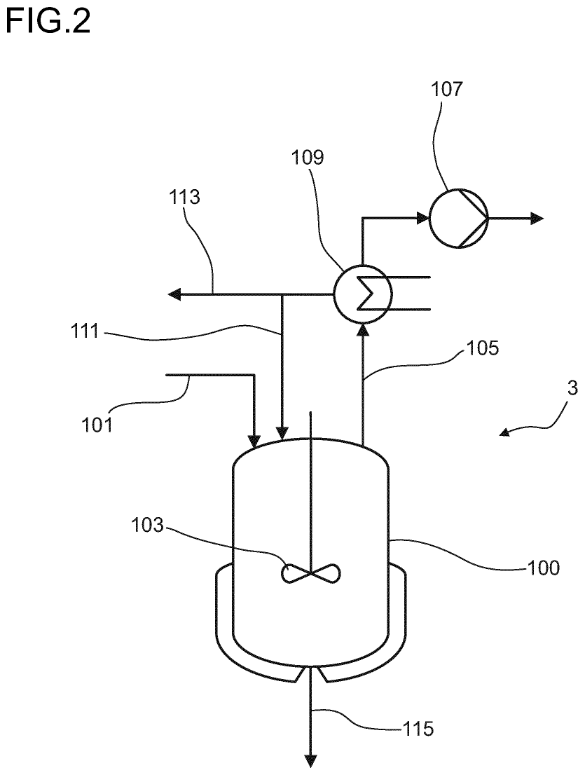

FIG. 2 a vessel for crystallization of DCDPSO.

An embodiment of the inventive process for obtaining DCDPSO from a liquid mixture comprising DCDPSO and solvent is shown in FIG. 1.

A liquid mixture 1 comprising DCDPSO and solvent is fed into a crystallization step 3. In the crystallization step 3 the liquid mixture is cooled to a temperature below the saturation point of DCDPSO in the solvent. This has the effect that DCDPSO starts to crystallize and a suspension is formed comprising solid DCDPSO crystals in a liquid which contains solvent, DCDPSO which is not crystallized and liquid byproducts. This suspension is fed into a solid-liquid-separation step 5. By solid-liquid-separation, for example filtration, the solid DCDPSO crystals are separated from the liquid phase, obtaining DCDPSO crystals 7 as product and mother liquor.

The solid-liquid-separation step 5 can be carried out in any suitable apparatus, particularly in a filtration apparatus, for example an agitated pressure nutsche, a rotary pressure filter, a drum filter or a belt filter or a centrifuge. The differential pressure in the filtration apparatus preferably is in the range between 100 and 6000 mbar, more preferred between 300 and 2000 mbar and particularly in the range between 400 and 1500 mbar. The filtration preferably is carried out at ambient temperature. Due to the necessary differential pressure in the filtration step, ambient pressure either can be set on the feed side which means that the pressure on the filtrate side is below ambient pressure, or ambient pressure is set on the filtrate side and a pressure above ambient pressure is set on the feed side.

The solid DCDPSO 7 is removed from the process and the mother liquor is fed into a concentrating step 9. In the concentrating step 9, solvent is removed from the mother liquor and withdrawn from the process as stream 11.

To remove by-products and impurities from the process which are not removed with the solvent, a part of the concentrated mother liquor is withdrawn as stream 13. The rest 15 of the concentrated mother liquor is recycled into the crystallization step 3.

The concentrating step 9 for example is a distillation or evaporation. In the distillation or evaporation solvent as low boiler is removed in gaseous form and the concentrated mother liquor containing the high boilers is removed in liquid form. If the mother liquor is concentrated by evaporation or distillation, the distillation or evaporation preferably is carried out at a pressure in the range between 20 and 800 mbar(abs), more preferred in a range between 50 and 500 mbar(abs), and particularly in a range between 100 and 350 mbar(abs). The bottom temperature if the concentrating step is operated by distillation or the temperature for evaporation preferably is in the range between 40 and 110° C., more preferred in the range of 55 and 100° C. and in particularly in the range between 55 and 80° C.

FIG. 2 shows a vessel for cooling and crystallizing DCDPSO.

To avoid fouling on cooled surfaces of a cooling and crystallization apparatus, it is preferred to use a gastight closed vessel 100 as shown in FIG. 2 for carrying out cooling and crystallization of DCDPSO. The cooling is performed by pressure reduction and the lowering of the boiling point due to the reduced pressure.

The liquid mixture comprising DCDPSO and solvent is fed into the vessel 100 via feed line 101. To achieve a homogeneous temperature and concentration in the liquid in vessel 100, the vessel 100 preferably is a stirred tank comprising at least one stirrer 103. By stirring the liquid mixture in the vessel further crystallized DCDPSO is kept in the forming dispersion and precipitation of crystallized DCDPSO and thus fouling is avoided.

For cooling the liquid mixture in the vessel 100 by dropping the boiling point of the liquid mixture due to pressure reduction, an exhaust gas line 105 is provided which is connected to a vacuum pump 107. A suitable vacuum pump 107 for example is a liquid ring pump, vacuum steam jet pump or steam jet ejector. Between the vessel 100 and the vacuum pump 107, a condenser 109 is accommodated in the exhaust gas line 105. In the condenser 109 solvent which is evaporated from the boiling liquid mixture in the vessel 100 is condensed by cooling. The condensed solvent then is returned into the vessel 100 via line 111. Further, to remove low boilers from the crystallization or to increase the concentration of DCDPSO in the liquid mixture to facilitate crystallization and thus increase the yield of solid DCDPSO obtained by crystallization, a withdrawing line 113 is provided via which condensed solvent and low boilers, if present, can be removed from the process.

By a drain line 115 suspension comprising crystallized DCDPSO is withdrawn from the vessel 100. The drain line 115 is connected to the filtration step 5 to feed the suspension into the filtration step 5.

The vessel 100 for cooling and crystallization can be operated either batchwise or continuously. If the vessel 100 for cooling and crystallization is operated batchwise, in a first step the liquid mixture is fed into the vessel 100. After a predefined filling level is reached, feeding of the liquid mixture is stopped. In a next step, the pressure in the vessel 100 is reduced using the vacuum pump 107 until a pressure in the vessel 100 is reached at which the boiling point of the liquid mixture is in a range between 80 and 95° C. Due to pressure reduction the liquid mixture starts boiling and solvent and low boilers evaporate. Once the saturation point of the DCDPSO in the solvent is reached, the pressure in the vessel is increased and the liquid mixture is heated to a temperature between 85 and 100° C. to dissolve partially the DCDPSO to achieve crystal nuclei of a homogeneous size. After this heating phase, the pressure in the vessel 100 is reduced again. By this pressure reduction the boiling point of the liquid mixture drops, solvent evaporates and is withdrawn from the vessel 100 via exhaust gas line 105. In the condenser 109 the evaporated solvent is condensed by cooling and the condensed solvent is recycled into the vessel 100. This recycling of solvent results in cooling of the liquid mixture leading to crystallization of DCDPSO. The temperature reduction in the vessel by pressure reduction and evaporation of the liquid is continued until the temperature in the vessel is in the range between 10 and 30° C., preferably ambient temperature.

After this temperature is reached, the pressure in the vessel is increased until ambient pressure is reached without heating the liquid mixture. Therefore, the suspension produced in the vessel 100 preferably has ambient temperature and ambient pressure before it is withdrawn from the vessel 100 via drain line 115.

By this process for cooling the liquid in the vessel 100 no cooled surfaces have to be provided on which DCDPSO would crystallize. Therefore, during crystallization no solid deposits on walls are formed.

If the vessel 100 is operated continuously, liquid mixture is continuously fed into the vessel 100 via feed line 101 and suspension comprising crystallized DCDPSO and solvent is continuously removed from the vessel 100 via drain line 115. In a continuous process preferably at least two vessels 100 connected into series are used. In the first vessel 100 the pressure in the vessel is kept constantly at a value at which the temperature is in a range from 65 to 85° C. and in the last vessel the pressure is kept such that the temperature is in the range from 0 to 45° C. If more than two vessels are used, the pressure in the vessels between the first and the last vessel is between the temperature in the first and in the last vessel and the temperature in all vessels decreases from the first to the last vessel. In each vessel 100 the temperature is set by withdrawing evaporated solvent via the exhaust gas line 105, condensing the evaporated solvent in the condenser 109 by cooling and returning the condensed solvent into the vessel 100 via line 111.

To keep a constant gas flow into the condenser 109 for continuous operation it is preferred to place an additional pump into the exhaust gas line 105 between the vessel 100 and the condenser 109 or into the line 111 between the condenser 109 and the vessel 100.

### EXAMPLES

Influence of final crystallization temperature (without concentrating and recycling of mother liquor) on the 4,4′-dichlorodiphenyl sulfoxide yield

A liquid mixture comprising 25 wt % DCDPSO based on the total amount of the liquid mixture was cooled to a desired temperature according to Table 1 at a cooling rate of 15 K/h by which a suspension formed. The suspension was filtrated to obtain a filter cake. The filter cake was washed with monochlorobenzene (100 g) and dried at 80° C. and 20 mbar(abs) overnight which yielded the desired product 4,4′-dichlorodiphenyl sulfoxide (4,4′-DCDPSO) as a fine white crystalline powder (1st isolated yield).

The mother liquor was distilled and concentrated to 20 wt % of the original amount. The concentrated mother liquor was cooled to 20° C. by which a suspension formed. This suspension was filtered to obtain a filter cake which was washed with chlorobenzene (100 g) and dried at 80° C. and 20 mbar(abs) overnight which yielded the desired product 4,4′-DCDPSO as a fine white crystalline powder (2nd isolated yield).

TABLE 1
Variation of crystallization temperatures

| | Crystallization | 1^{st} and 2^{nd} | Purity |
|---|---|---|---|
| | temperature | Isolated | 4,4′-DCDPSO |
| Experiment | [° C.] | Yield [%] | [wt %] |
| 1 | 0 | 78.3/n.d. | 100/— |
| 2 | 20 | 77.9/5.5 | 100/92 |
| 3 | 30 | 77.9/7.7 | 100/95 |
| 4 | 40 | 74.5/11.5 | 99.5/96 |

Crystallization without concentrating and recycling the mother liquor (comparative example)

A liquid mixture comprising 25.2 wt % DCDPSO, 72.9 wt % monochlorobenzene, 0.2 wt % 4,4′-dichlorodiphenylsulfide and 1.7 wt % 2,4′-dichlorodiphenylsulfoxide which was obtained in a reaction for obtaining DCDPSO was subjected to a distillation. Monochlorobenzene was distilled from the liquid mixture until saturation was reached at about 88° C. (monitored via a turbidity probe, distillation conditions: 200 mbar(abs)). Then the liquid mixture was cooled by reducing the pressure until the temperature reached 30° C. By the cooling a suspension comprising crystallized DCDPSO was obtained which was objected to a filtration process to obtain a filter cake comprising crystallized DCDPSO.

After filtration and washing of the filter cake with monochlorobenzene the crystalline solid was dried at 100° C. and 100 mbar(abs).

The 4,4′-dichlorodiphenyl sulfoxide was obtained in 83.2% yield, with a purity of 98.8 wt %, containing 0.6 wt % monochlorobenzene, 0.2 wt % 4,4′-dichlorodiphenylsulfide and 0.4 wt % 2,4′-dichlorodiphenylsulfoxide.

Influence of concentrating and recycling the mother liquor into the crystallization process (inventive example)

A liquid mixture comprising 26.7 wt % DCDPSO, 66.3 wt % monochlorobenzene, 0.5 wt % 4,4′-dichlorodiphenylsulfide and 6.5 wt % 2,4′-dichlorodiphenylsulfoxide which was obtained in a reaction for obtaining DCDPSO was subjected to a distillation. Monochlorobenzene was distilled from the liquid mixture until saturation was reached at about 88° C. (monitored via a turbidity probe, distillation conditions: 200 mbar(abs)). Then the liquid mixture was cooled by reducing the pressure until the temperature reached 30° C. By the cooling a suspension comprising crystallized DCDPSO was obtained which was objected to a filtration process to obtain a filter cake comprising crystallized DCDPSO.

After filtration and washing of the filter cake with monochlorobenzene the crystalline solid was dried at 100° C. and 100 mbar(abs). The combined mother liquor and washing filtrate were subjected to a distillation. In the distillation monochlorobenzene was removed until the amount of combined mother liquor and washing filtrate was reduced to 25 wt %. The distillation was operated at a bottom temperature of 90° C. and 200 mbar(abs).

80 wt % of the obtained bottom product were transferred into the crystallization of the next batch.

The 4,4′-dichlorodiphenyl sulfoxide yield in the steady state with loop crystallization were 1232 g which corresponds to 91.3%.

The 4,4′-dichlorodiphenyl sulfoxide had a purity of 98.9 wt %, containing 0.5 wt % monochlorobenzene, 0.3 wt % 4,4′-dichlorodiphenylsulfide and 0.3 wt % 2,4′-dichlorodiphenylsulfoxide.

1.-14. (canceled)

15. A process for obtaining 4,4′-dichlorodiphenyl sulfoxide from a liquid mixture comprising 4,4′-dichlorodiphenyl sulfoxide and chlorobenzene, comprising:
(a) cooling the liquid mixture to a temperature below the saturation point of 4,4′-dichlorodiphenyl sulfoxide in the chlorobenzene to obtain a suspension comprising crystallized 4,4′-dichlorodiphenyl sulfoxide,
(b) solid-liquid-separation of the suspension to obtain a residual moisture containing solid 4,4′-dichlorodiphenyl sulfoxide as a product and mother liquor,
(c) concentrating the mother liquor,
(d) recycling at least a part of the concentrated mother liquor into the cooling step (a).

16. The process according to claim 1, wherein the chlorobenzene is monochlorobenzene.

17. The process according to claim 1, wherein cooling step (a) is carried out in a gastight closed vessel (100) by
(i) reducing the pressure in the gastight closed vessel (100);
(ii) evaporating solvent;
(iii) condensing the evaporated solvent by cooling; and
(iv) returning the condensed solvent into the gastight closed vessel.

18. The process according to claim 17, wherein steps (ii) to (iv) are carried out during pressure reduction in step (i).

19. The process according to claim 17, wherein the pressure reduction in step (i) is continued until the pressure in the gastight closed vessel (100) reaches a predefined value in the range between 20 to 350 mbar(abs).

20. The process according to claim 17, wherein after the pressure reached the predefined value the process is finished, and the pressure is set to ambient pressure.

21. The process according to claim 17, wherein for initializing crystallization of the 4,4′-dichlorodiphenyl sulfoxide following steps are carried out before setting the reduced pressure in step (i):
reducing the pressure in the gastight closed vessel such that the boiling point of the mixture is in the range between 80 and 95° C.;
evaporating solvent until an initial formation of solids takes place;
increasing the pressure in the vessel and heating the liquid mixture in the vessel to a temperature in the range between 85 and 100° C.

22. The process according to claim 15, wherein the mother liquor is concentrated by distillation or evaporation of solvent.

23. The process according to claim 22, wherein the distillation or evaporation is carried out at a pressure in the range between 20 and 800 mbar(abs).

24. The process according to claim 22, wherein the distillation is carried out in a distillation column with a bottom temperature in a range from 40 to 110° C. and a head temperature in a range from 30 to 100° C.

25. The process according to claim 22, wherein the evaporation or distillation is continued until the amount of mother liquor is reduced to 4 to 80 wt. % of the amount of mother liquor fed into the evaporation or distillation.

26. The process according to claim 22, wherein the evaporation or distillation is continued until the concentration of 4,4′-dichlorodiphenyl sulfoxide in the mother liquor is in the range from 6 to 60 wt. %.

27. The process according to claim 15, wherein the amount of concentrated mother liquor recycled into the cooling (a) is in the range from 10 to 95 wt. %.

28. The process according to claim 15, wherein the cooling (a) is continued until a solids content in the suspension in the range from 5 to 50 wt. % is achieved.
